(19) United States
(12) Patent Application Publication
(10) Pub. No.: US 2009/0221852 A1
(43) Pub. Date: Sep. 3, 2009

(54) Preparation of an Atorvastatin Intermediate

(75) Inventors: Susan O'Sullivan, Cork (IE); Elizabeth Fox, Cork (IE)

Correspondence Address: PFIZER INC.;PATENT DEPARTMENT, Bld 114 M/S 114, EASTERN POINT ROAD, GROTON, CT, 06340, US

(73) Assignee: Pfizer Inc.

(21) Appl. No.: 12/065,546

(22) PCT Filed: Sep. 9, 2005
(86) PCT No.: PCT/IE05/00095

§ 371 Date: Oct. 20, 2008

(51) Int. Cl. C07C 235/80

(52) U.S. Cl. 564/169

(57) ABSTRACT

The diketone of atorvastatin is prepared by first washing a reaction vessel with a non-ketonic solvent, especially tetrahydrofuran, to remove water. 4-fluorobenzaldehyde is then reacted with benzylidine isobutyryl acetanilide in the reaction vessel to form 4-fluoro-alpha-(2-methyl-1-oxopropyl)-gamma-oxo-N,beta-diphenylbenzene-butanainide

The invention relates to a process for preparing the diketone of atorvastatin which is a key intermediate in the preparation of atorvastatin lactone. Atorvastatin lactone is a trans-6-[2-(substituted pyrrole-1-yl)alkyl]pyran-2-one which is known by the chemical name (2R-trans)-5-(4-fluorophenyl)-2-(1-methyethyl)-N,4-diphenyl-1-[2-(tetrahydro-4-hydroxy-6-oxo-2H-pyran-2-yl)ethyl]-1H-pyrrole-3-carboxamide.

Atorvastatin lactone is the penultimate intermediate in the preparation of another trans-6-[2-(substituted pyrrole-1-yl)alkyl]pyran-2-one, atorvastatin calcium known by the chemical name [R—R*,R*)]-2-4fluorophenyl-β,δ-dihydroxy-5-methylethyl)-3-phenyl-4-[(phenylamino)carbonyl]-1H-pyrrole-1-heptanoic acid hemi calcium salt.

Atorvastatin as well as some of its metabolites is pharmacologically active in humans and is useful as a hypolipidemic and hypocholesterolemic agent. In particular, atorvastatin is useful as a selective and competitive inhibitor of the enzyme 3-hydroxy-3-methylglutaryl-coenzyme A (HMO-CoA) reductase, the rate-limiting enzyme that converts 3-hydroxy-3-methylglutaryl-coenzyme A to mevalonate, a precursor of sterols such as cholesterol. The conversion of HMG-CoA to mevalonate is an early and rate-limiting step in cholesterol biosynthesis.

U.S. Pat. No. 4,681,893, which is herein incorporated by reference, discloses certain trans- 6-[2-(3- or 4-carboxamido-substituted-pyrrol-1-yl)alkyl)]4-hydroxy-pyran-2-ones including trans (±)-5-(4fluorophenyl)-2-(1-methylethyl)-N,4-diphenyl-1-[(2-tetrahydro-4-hydroxy-6-oxo-2H-pyran-2-yl)ethyl]1H-pyrrole-3-carboxamide.

U.S. Pat. No. 5,273,995, which is herein incorporated by reference, discloses the enantiomer having the R form of the ring-opened acid of trans -5-(4-fluorophenyl)-2-(1-methylethyl)-N,4diphenyl-1-[(2-tetrahydro-4-hydroxy-6-oxo-2H-pyran-2-yl)ethyl]-1H-pyrrole-3-carboxamide, i.e., [R-(R*,R*)]-2-(4-fluorophenyl)-β, δ-dihydroxy-5-(1-methylethyl)-3-phenyl-4-[(phenylamino) carbonyl]-1H-pyrrole-1-heptanoic acid.

The above described atorvastatin compounds have been prepared by a superior convergent route disclosed in the following U.S. Pat. Nos. 5,003,080; 5,097,045; 5,03,024; 5,124,482 and 5,149,837 which are herein incorporated by reference and Baumann K. L., Butler D. E., Deering C. F., et al, Tetrahedron Letters 1992;33:2283-2284.

One of the critical intermediates outlined in U.S. Pat. No. 5,097,045 has also been produced using novel chemistry, as described in U.S. Pat. No. 5,155,251, which is herein incorporated by reference and Brower P. L., Butler D. E., Deering C. F., et al, Tetrahedron Letters 1992;33:2279-2282.

U.S. Pat. Nos. 5,216,174; 5,245,047; 5,248,793; 5,280,126; 5,397,792; 5,342,952; 5,298,627; 5,446,054; 5,470,981; 5,489,690; 5,489,691; 5,5109,488; WO97/03960; WO98/09543 and WO99/32434 which are herein incorporated by reference, disclose various processes and key intermediates for preparing atorvastatin.

The process for preparing atorvastatin intermediates is particularly sensitive and vulnerable to the formation of process impurities which may cause product rejection and decreased yields.

The object of the present invention is therefore to provide an improved process for preparing atorvastatin intermediates in which the formation of reaction impurities is minimised.

### STATEMENTS OF INVENTION

According to the present invention there is provided a process for the production of the diketone of atorvastatin comprising the steps of:
washing a reaction vessel with a non-ketonic solvent to remove water; and
reacting 4-fluorobenzaldehyde with benzylidine isobutyryl acetanilide in the reaction vessel to form 4-fluoro-alpha-(2-methyl-1-oxopropyl)-gamma-oxo-N,beta-diphenylbenzenebutanamide according to the following reaction scheme:

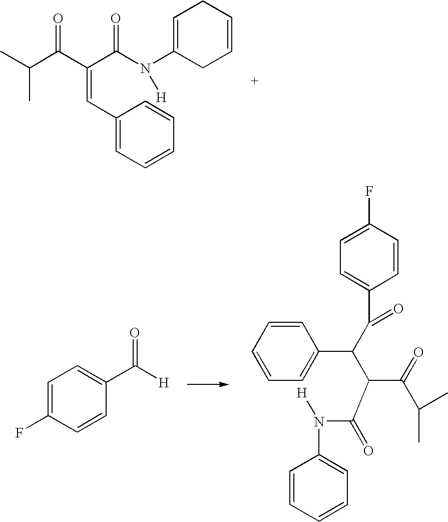

Preferably the non-ketonic solvent is tetrahydrofuran.

In one embodiment the process includes the step of collecting the wash-off material and discharging it from the vessel prior to the introduction of the reactants.

In a further embodiment the non-ketonic solvent is introduced into the reaction vessel through a spray ball to substantially cover all of the inner surfaces of the vessel. The reaction vessel includes an agitator which is also dried by the non-ketonic solvent.

### DETAILED DESCRIPTION

The invention will be more clearly understood from the following description thereof given by way of example only.

The diketone of atorvastatin or 4-fluoro-alpha-(2-methyl-1-oxopropyl)-gamma-oxo-N,beta-diphenylbenzenebutanamide, is prepared in a single step by the reaction of 4-fluorobenzaldehyde with 2-benzylidine isobutyryl acetanilide in a Stetter reaction as shown in scheme 1.

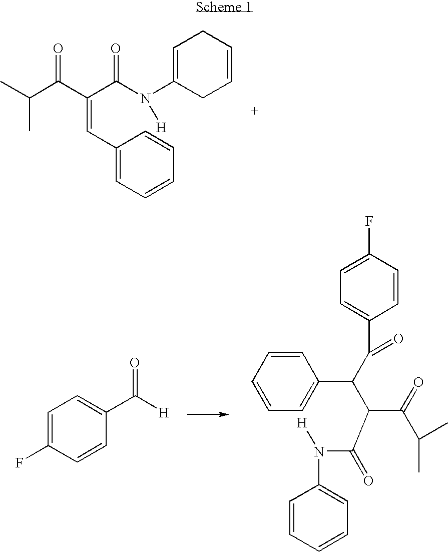

Impurities which have been detected in the diketone of atorvastatin arise from unreacted starting material, impurities derived from starting material contaminants and reaction by-products. The process is particularly sensitive to the presence of trace amounts of water which can cause the formation of several process impurities such as desfluoro diketone.

We have found that by washing and drying the reaction equipment with a non-ketonic solvent a significant reduction in impurity formation can be achieved. Previously the reaction equipment was washed with acetone which is the solvent of choice for such applications in view of its ready availability, relatively low cost, low boiling point and water miscibility.

However, we have found that even trace amounts of acetone react with the aldehyde starting material to form water. The presence of water in turn encourages the formation of undesirable desfluro diketone impurities.

The desfluro diketone impurity is of very similar structure to the desired diketone as will be apparent from scheme 2.

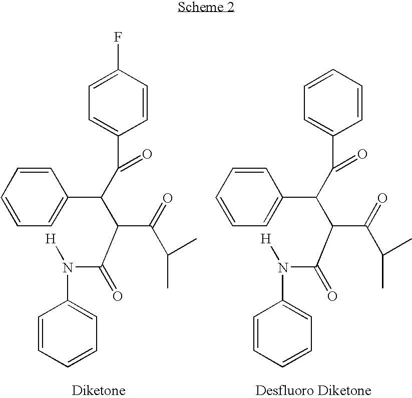

While other impurities may be readily removed from the reaction process using recrystallisation, the desfluro diketone impurity is especially problematic. If the desfluro impurity is above 0.45% the product has failed and cannot be recovered. If the desfluro impurity is less than about 0.45% it may be possible to recover the product after one or more recrystallisation steps.

Tetrahydrofuran (THF) was found to be the ideal non-ketonic solvent as at ambient temperature it dissolves diketone with the advantage of no adverse effect on the reaction as it is already used as a reaction solvent. It is also a dry solvent with a low water specification of 0.03%.

The present invention therefore provides an improved process for the preparation of the diketone of atorvastatin, a key intermediate in the preparation of atorvastatin lactone.

### EXAMPLE 1

### Preparation of 4-fluoro-alpha-[2-methyl-1-oxopropyl]-gamma-oxo-N,beta-diphenylbenzenebutanamide

A reaction vessel is inerted using at least 4 cycles of vacuum, releasing the vacuum each time with nitrogen. 250 litres of tetrahydrofuran is charged to the reaction vessel via spray nozzles. Spray ball nozzles ensure that all areas of the reaction vessel are penetrated in particular the top inner surface of the vessel and the agitator device also present inside the reaction vessel. The tetrahydrofuran washings are drained off and collected for waste recycling.

When the reaction vessel is dry 480 kgs 2-benzylidine isobutyrylacetamide (BIBEA), 60 kgs ethyl hydroxyethylmethyl thiazolium bromide (MTB or ethyl hydroxyethyl MTB), 200 litres, 216 kgs of 4-fluorobenzaldehyde and 120 kgs of triethylamine are charged to the reaction vessel and heated with agitation to between 60 and 70° C. The reaction mixture is aged for 16 to 24 hrs maintaining the temperature at 65+/−5° C. The contents are then cooled to 60+/−5° C. for 54 to 66 minutes. 600 litres of isopropanol is charged to the reaction mixture and the mixture is heated to about 100° ° C. to achieve a solution.

600 litres of deionised water is charged to the reaction vessel over 30 minutes while maintaining the temperature at 60+/−5° C. The batch is aged for 54 to 66 minutes and the contents cooled to between 25+/−5° C. over a 2 to 4 hour period at a rate of 15/20° C. per hour. The batch is aged at this temperature for at least 1 hour and the contents cooled further to 0+/−5° C. and aged for at least 1 hour.

The batch is isolated on a filter and washed with isopropanol. The product is dried under-vacuum at 50+/−5° C. to a water content of less than 0.5%. The contents are then cooled to approximately less than 30° C. before discharging.

The invention is not limited to be embodiments hereinbefore described which may be varied in detail.

1. A process for the production of the diketone of atorvastatin comprising the steps of :
washing a reaction vessel with a non-ketonic solvent to remove water; and
reacting 4-fluorobenzaldehyde with benzylidine isobutyryl acetanilide in the reaction vessel to form 4-fluoro-alpha-(2-methyl-1-oxopropyl)-gamma-oxo-N,beta-diphenylbenzenebutanamide according to the following reaction scheme:

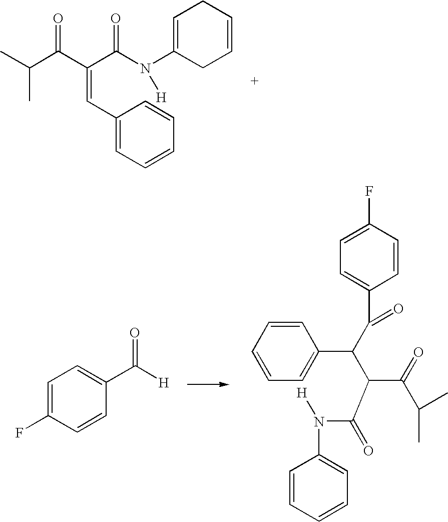

2. A process as claimed in claim 1 wherein the non-ketonic solvent is tetrahydrofuran.

3. A process as claimed in claim 1 or 2 including the step of collecting the wash-off material and discharging it from the vessel prior to the introduction of the reactants.

4. A process as claimed in any preceding claim wherein the non-ketonic solvent is introduced into the reaction vessel through a spray ball to substantially cover all of the inner surfaces of the vessel.

5. A process as claimed in any preceding claim wherein the reaction vessel includes an agitator which is also dried by the non-ketonic solvent.
